(19) United States
(12) Patent Application Publication
(10) Pub. No.: US 2014/0271475 A1
(43) Pub. Date: Sep. 18, 2014

(54) CELLULOSE NANOPARTICLE BIODEGRADABLE PHOTOACOUSTIC CONTRAST AGENTS

(71) Applicant: The Board of Trustees of the Leland Stanford Junior University, Palo Alto, CA (US)

(72) Inventors: Jesse Jokerst, San Francisco, CA (US); Sanjiv S. Gambhir, Portola Valley, CA (US)

(21) Appl. No.: 14/171,087

(22) Filed: Feb. 3, 2014

Related U.S. Application Data
(60) Provisional application No. 61/776,894, filed on Mar. 12, 2013.

(51) Int. Cl. A61K 49/22

(52) U.S. Cl. CPC A61K 49/225
USPC 424/9.1; 428/402

(57) ABSTRACT

Biodegradable cellulose photoacoustic probes are provided that are able to detect biological targets and generate a detectable photoacoustic signal. The cellulose nanoparticles are biodegradable by cellulases. Clearance of the nanoparticles from a subject is enhanced and results in improved contrast of a photoacoustic-generated image. Methods to generate images by administering a cellulose nanoparticle probes to a subject and irradiating with light of a wavelength to emit photoacoustic energy detectable by sensors external to the subject are also provided. The detectable signal is convertible to a visual image of the nanoparticle probes in the subject. The methods may include allowing a cellulase to degrade nanoparticles so that undesirable background signals emitted are substantially reduced to increase the contrast and quality of the generated image. In the absence of an endogenous source of an enzyme for degrading the cellulose nanoparticles, a composition comprising a heterologous cellulase may be administered along with the nanoparticles.

### CROSS-REFERENCE TO RELATED APPLICATION

This application claims priority to U.S. Provisional Application No. 61/776,894, entitled “CELLULOSE NANOPARTICLE BIODEGRADABLE PHOTOACOUSTIC CONTRAST AGENTS” filed on Mar. 12, 2013, the entirety of which is herein incorporated by reference in its entirety.

### STATEMENT ON FUNDING PROVIDED BY THE U.S. GOVERNMENT

This invention was made with Government support under Contracts CA118681, CA151459, and CA114747, awarded by the National Institutes of Health. The Government has certain rights in this invention.

### TECHNICAL FIELD

The present disclosure is generally related to cellulose-based nanoparticles suitable for photoacoustic imaging of isolated cells or population of cells, or tissue in an animal or human subject. The present disclosure is further related to methods of their use in the photoacoustic imaging.

### BACKGROUND

Photoacoustic imaging (PAI) has found broad applications in oncology including prostate (Agarwal et al., (2007) J. Appl. Phys. 102: 064701-064704), breast (Manohar et al., (2004) J. Biomed. Opt. 9: 1172-1181), and ovarian cancer (Aguirre et al., (2011) Transl. Oncol. 4: 29-37). This modality produces contrast by converting nanosecond light pulses into an acoustic signal and offers very significant improvements in spatial resolution relative to an optics-based approach (Xu & Wang (2006) Rev. Sci. Instr. 77: 041101; Wang & Hu (2012) Science 335: 1458-1462; Ntziachristos & Razansky (2010) Chem. Rev. 110: 2783-2794). Ovarian cancer in particular could benefit from PAI due to the existing widespread use of ultrasound in the screening and management of this disease (Lutz et al., (2011) Radiology 259: 329-345; Bast et al., (2009) Nat. Rev. Cancer 9: 415-428). PAI can use either an endogenous signal from hemoglobin, deoxyhemoglobin, melanin, etc., or an exogenous imaging agent can be administered. For most molecular imaging applications, the introduction of an exogenous imaging agent is particularly advantageous when adapted to selectively target a cell population or specific cell component

Many materials produce photoacoustic signal, and these can be broadly grouped as small molecules and nanoparticles. Small molecule agents include dyes such as methylene blue or indocyanine green, fluorophores (including fluorescent proteins) (Razansky et al., (2009) Nature Photon. 3: 412-417), quenchers (van de Ven et al., (2011) Mol. Imaging Biol. 13: 232-238), and activatable hybrid molecules (Levi et al., (2010) J. Am. Chem. Soc. 132: 11264-11269;). Nanoparticle-based imaging uses gold nanoparticles, gold/silica hybrids (Kircher et al., (2012) Nat. Med. 18: 829-834; Chen et al., Nano Lett. 11: 348-54), carbon nanotubes (De la Zerda et al., (2008) Nature Nanotech. 3: 557-562), porphysomes (Lovell et al., (2011) Nat. Mater. 10: 324-332), iron oxide nanoparticles, copper sulfide, and the like (Zerda et al., (2011) CMMI 6: 346-369; Kim et al., (2007) J. Biomed. Opt. 12: 044020; Wilson et al., (2012) Nature Comm. 3: 618).

The main limitation of small molecules, however, is their poor photoacoustic signal. Although nanoparticles offer robust and intense photoacoustic signals, they are hampered by poor biodistribution and clearance profiles. Indeed, one of the most common limitations of all nanoparticle imaging agents is non-specific, long term liver and spleen accumulation. While porphysomes (Lovell et al., (2011) Nat Mater 10: 324-332) and plasmonic nanoclusters containing 5 nm gold particles cross-linked for red-shifted resonances (Tam et al., (2010) ACS nano 4: 2178-2184; Yoon et al., (2010) Optics letters 35: 3751-3753) may offer renal clearance, their full utility in small animal models remains unclear. Any agent that combines the signal intensity of nanoparticles with the renal clearance of small molecules would have a significant advantage towards clinical translation.

### SUMMARY

Molecular imaging with photoacoustic ultrasound is an emerging field that combines the spatial and temporal resolution of ultrasound with the contrast of optical imaging. However, there are few imaging agents that offer both high signal intensity and obvious clearance/biodegradation profiles. The present disclosure provides a cellulose-based nanoparticle with a photoacoustic signal at about 700 nm that has an approximately 2.5-fold higher signal than gold nanorods at the same concentration. The cellulose nanoparticles of the disclosure were shown to biodegrade in the presence of cellulase, thereby facilitating their removal via the renal system.

One aspect of the disclosure, therefore, encompasses embodiments of a method of generating a detectable photoacoustic signal in a biological subject, the method comprising: (i) administering to a biological subject a first pharmaceutically acceptable composition comprising a degradable cellulose nanoparticle probe that generates a detectable photoacoustic signal when exposed to an excitation optical energy; (ii) allowing the probe to concentrate in a cell or tissue of the biological subject; (iii) allowing degradation of the administered cellulose nanoparticle probes not concentrated in the cell or tissue; (iv) illuminating the recipient subject with an incident energy having a wavelength that generates an emitted photoacoustic signal from the concentration of cellulose nanoparticle probes; (v) detecting the emitted photoacoustic signal; and (vi) determining at least one of the presence and the location of the emitted detectable photoacoustic signal in the biological subject.

In some embodiments of this aspect of the disclosure, the method further comprises allowing the cellulose nanoparticle probes degrade after step (v).

In embodiments of this aspect of the disclosure, the method may further comprise generating from the detectable emitted photoacoustic signal an image of the concentration of the cellulose nanoparticle probes in the biological subject.

In embodiments of this aspect of the disclosure, the biological subject receiving the administered pharmaceutically acceptable composition can be an isolated cell, a cultured cell, an isolated tissue, or a tissue or region of an animal or human subject.

In embodiments of this aspect of the disclosure, the degradation of the cellulose nanoparticle probes not concentrated in a target cell or tissue of the recipient subject can increase the photoacoustic signal to background signal ratio.

In embodiments of this aspect of the disclosure, the cellulose nanoparticle probes can be degraded by an endogenous cellulase activity.

In some embodiments of this aspect of the disclosure, the method can further comprise delivering to the recipient subject a second pharmaceutically acceptable composition, wherein said composition can comprise a cellulase. The cellulase delivered to the recipient subject can be in an amount effective for the degradation of the cellulose nanoparticle probes not concentrated in target cell or tissue of the recipient subject.

In some embodiments of this aspect of the disclosure, the first and the second pharmaceutically acceptable compositions can be administered simultaneously to the recipient subject. In other embodiments of this aspect of the disclosure, the first and the second pharmaceutically acceptable compositions can be administered consecutively to the recipient subject.

In embodiments of this aspect of the disclosure, the photoacoustic cellulose nanoparticles can have a fluorescent dye attached thereto.

In embodiments of this aspect of the disclosure, the photoacoustic cellulose nanoparticles can be adapted to selectively bind to a target cell or tissue by having a target-specific moiety attached thereto.

In some embodiments of this aspect of the disclosure, the photoacoustic cellulose nanoparticles can be derived from cotton linter.

Another aspect of the disclosure encompasses embodiments of a degradable cellulose nanoparticle probe, wherein said nanoparticle can have an amorphous and anisotropic conformation having an average dimension of about 25 nanometers (nm) to about 250 nm.

In some embodiments of this aspect of the disclosure, the probe can absorb energy at about 700 nm.

In some embodiments of this aspect of the disclosure, the cellulose nanoparticle can have a diameter of about 130±45 nm.

In embodiments of this aspect of the disclosure, the nanoparticle can further comprise a dye compound or plurality of dye compounds attached to the cellulose nanoparticle.

In embodiments of this aspect of the disclosure, the dye can be selected from the group consisting of: a diarylrhodamine derivative, a polyaromatic-azo quencher, Blackberry Q, and a bisazulene derivative, Black Hole quenchers, and the like.

In embodiments of this aspect of the disclosure, the degradable cellulose nanoparticle probe can further comprise a targeting moiety bound to the cellulose nanoparticle.

In embodiments of this aspect of the disclosure, the targeting moiety can be bound to the cellulose nanoparticle via a linker molecule.

Some embodiments of this aspect of the disclosure encompass cellulose nanoparticles generated by the method of disclosure.

Still another aspect of the disclosure encompasses embodiments of a method of generating a degradable cellulose nanoparticle probe, the method comprising the steps of: contacting an aqueous suspension of cellulose fibers with about 6M sulfuric acid; cooling said acidified suspension to ambient temperature over a period of between about 2 h to about 4 h; fractionating the acidified suspension by centrifugation; resuspending a pelleted fraction from the centrifugation in water; and dialyzing the resuspended pellet with a dialysis membrane having a molecular weight cutoff value between about 3000 daltons to about 4000 daltons, thereby obtaining a suspension of cellulose nanoparticles having an amorphous and anisotropic conformation with an average dimension of about 25 nanometers (nm) to about 250 nm.

In embodiments of this aspect of the disclosure, the cellulose fibers can be from cotton linter.

### BRIEF DESCRIPTION OF THE DRAWINGS

Aspects of the present disclosure will be more readily appreciated upon review of the detailed description of its various embodiments, described below, when taken in conjunction with the accompanying drawings. The drawings are described in greater detail in the description and examples below.

FIGS. 1A-1E illustrate the determination of the physical characteristics of cellulose-based nanoparticles. Cellulose nanoparticles (CNPs) were examined with TEM imaging at increasing magnification (FIGS. 1A-1C) and showed amorphous and anisotropically-shaped nanoparticles with dimensions of about 132±46 nm. Panels in FIGS. 1A and 1B indicate the subsequent higher magnification image in panels of FIGS. 1C and 1D. The hydrodynamic radius as determined by dynamic light scattering (DLS) was 196.0 nm with a polydispersity index (PDI) of about 0.138. FIG. 1E illustrates the absorbance spectra of CNPs at 0.077 mg/mL (0.023 nM) in PBS showing Rayleigh scattering with maximum absorbance in the UV visible region (black line), contrasting to gold nanorods at 0.34 nM with a resonance tuned to 700 nm. The inset in FIG. 1E shows gold nanorods with dimensions of 40.0×13.2 nm that were used for signal comparisons.

FIGS. 2A-2C illustrate the photoacoustic signaling capacity of the CNPs of the disclosure.

FIG. 2A shows that spectral imaging of CNPs highlights the maximum absorbance peak at 700 nm. The background photoacoustic (PA) spectrum of normal tissue is also shown for reference. Inset is a photoacoustic image of a subcutaneously implanted bolus of CNPs suspended in matrigel at 0.5 mg/mL (“Pos.”) or a matrigel-only implant (“Neg.”). The solid circle highlights a normal region used to create the tissue-only spectrum. Scale bar in inset is 3 mm.

FIG. 2B illustrates representative photoacoustic imaging data of a phantom scanned with the tomographic imaging system. Panels Bi-Biv illustrate CNPs at the following concentrations: Bi, 0.70 nM; Bii, 0.35 nM; Biii, 0.15 nM; Biv, 0.07 nM. Panel Bv is 0.70 nM gold nanorods (GNRs). Scale bar represents 4 mm.

FIG. 2C illustrates photoacoustic intensity data collected with the tomographic scanner for both CNPs and GNRs with 700 nm incident radiation. Concentration values are given in molar units and weight units (mg/mL in italics). At an isomolar concentration of imaging agents, the CNPs produce about 2-3-fold more detectable PA signal. Error bars in FIG. 2C represent the standard deviation of 3 replicate samples and are less than 10% RSD.

FIGS. 3A and 3B illustrate toxicity data. Cultured OV2008 cells (n=6 replicate wells) were exposed to increasing concentrations of CNPs overnight and then analyzed with the Presto Blue reagent. For a positive control (“POS”), cetyltrimethylammonium bromide was used as a toxic agent to validate the PRESTOBLUE.RTM reagent and assay. The asterisk indicates a statistically significant change (p<0.05). Error bars in FIG. 3A represent the standard deviation of 6 replicates

FIG. 3B illustrates the results of serum electrolyte and liver function tests from animals treated with CNPs at increasing concentrations compared to untreated animals (Control). Filled columns indicate a value (p<0.05) statistically different from the control cohort. Aspartate transaminase (AST), alanine transaminase (ALT), and alkaline phosphatase (AlkPhos): U/L; Na, K, CO₂, anion gap, and K: mmol/L. Error bars in FIG. 3B represent the standard deviation of 3 animals.

FIGS. 4A-4F illustrate the biodegradation of CNPs upon treatment with cellulase.

FIG. 4A illustrates a hexokinase glucose assay validated with glucose standards in dilute acetic acid.

FIG. 4B illustrates the activity of the cellulase enzyme validated with a cellulose standard and that it caused an increased glucose concentration in samples with cellulase, but not the control (no cellulase) samples.

FIG. 4C shows both control (native cellulose not in nanoparticle form) and CNPs produced glucose in the presence of cellulase, suggesting biodegradation. Both cotton linter-derived CNPs (“CNP lin”) and Sigmacell (“CNP sig”) were studied. Error bars in FIGS. 4A-4C represent the standard deviation of at least 3 replicate measurements. Smaller fragments in the cellulase-treated CNPs are highlighted by arrows.

FIG. 4D shows TEM imaging of naïve CNPs.

FIG. 4E shows TEM imaging of CNPs treated with heat.

FIG. 4F shows TEM imaging of CNPs treated with heat/acid only.

FIG. 5A shows time-activity curves of CNP of the disclosure in a subcutaneous murine model of human ovarian cancer (OV2008 line). Three different animals were imaged before (0) and 1, 15, 30, 45, and 60 min after tail-vein injection of 200 μL of 2.4 mg/mL CNPs. The photoacoustic (PA) signal at any given time point was divided by the PA signal pre-injection to give the relative units graphed. The dashed line indicates no increase above baseline. All three animals showed maximum intensity 15-30 min after injection. This pattern was also seen at the other concentration values presented in FIG. 5B.

FIG. 5B illustrates the fold-increase above baseline plotted against those concentration values examined (statistically significant above baseline at p<0.05). Error bars represent the standard error for the three animals. The relationship between PA signal and concentration of injected contrast was linear at R²>0.96.

FIG. 6 shows imaging data. Dashed lines in panels A and Ai highlight the imaging plane used to create the renderings in panels B-E. Top images are before injection of CNPs and lower panels denoted by “i” are post-injection. B, no injection; C, 1.2 mg/mL; D, 2.4 mg/mL; E, 4.0 mg/mL. Intensity bar in panel E applies to all images as does the scale bar in panel Ei, which represents 3 mm. Arrows highlight regions with particularly increased PA contrast in post-injection images.

### DESCRIPTION OF THE DISCLOSURE

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “ includes,” “including,” and the like; “consisting essentially of”or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law, allowing for the presence of more than that which is recited so long as basic or novel characteristics of that which is recited is not changed by the presence of more than that which is recited, but excludes prior art embodiments.

### Abbreviations

PAI, photoacoustic imaging; PA, photoacoustic; CNP, cellulose nanoparticle; TEM, transmission electron microscopy

### Definitions

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

The term “acoustic detectable signal” is a signal derived from a probe of the present disclosure that absorbs light and converts absorbed energy into thermal energy that causes the generation of an acoustic signal through a process of thermal expansion. The “acoustic detectable signal” is detectable and distinguishable from other background acoustic signals that are generated from the host. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the acoustic detectable signal and the background) between acoustic detectable signal and the background. Standards and/or calibration curves can be used to determine the relative intensity of the acoustic detectable signal and/or the background.

The term “acoustic signal” as used herein refers to a sound wave produced by one of several processes, methods, interactions, or the like, that provides a signal that can then be detected and quantitated with regards to its frequency and/or amplitude. The acoustic signal can be generated from or modulated by one or more particles of the present disclosure. In an embodiment, the acoustic signal may be the sum of each of the individual ultrasound or photoacoustic signals. In an embodiment, the acoustic signal can be generated from a summation, integration, or other mathematical process, formula, or algorithm, where the acoustic signal is from one or more probes. In an embodiment, the summation, the integration, or other mathematical process, formula, or algorithm can be used to generate the acoustic signal so that the acoustic signal can be distinguished from background noise and the like. It should be noted that signals other than the acoustic signal can be processed or obtained is a similar manner as that of the acoustic signal.

The acoustic signal can be detected and quantified in real time using an appropriate detection system. For example, two instruments that can be used quantifying acoustic signal are the NEXUS128.RTM (Endra Life Sciences, Ann Arbor, Mich.), the VEVO.RTM 2100 (Fujifilm VisualSonics, Inc., Toronto, Canada). Others can be used and purchased from manufacturers such as iThera. The units of acoustic signal can vary and include echogenicity units (EU) or mean grey scale. Input units include dB and frequency (MHz). Maximum intensity persistence imaging can also be used and is described by Pysz et al. in Investigative Radiology (2011) 46: 187-195. Other detection strategies, including capacitive micromachined ultrasonic transducers (CMUT) arrays, can also be used to detect the acoustic signal.

The term “administration” as used herein refers to the introduction of an embodiment of a cellulose nanoparticle probe of the present disclosure into a subject. While the preferred route of administration of an embodiment of the present disclosure is intravenously, it is contemplated that any route of administration, such as oral, topical, subcutaneous, peritoneal, intra-arterial, inhalation, vaginal, rectal, nasal, introduction into the cerebrospinal fluid, or instillation into body compartments can be usefully employed in the methods of the disclosure.

The term “aqueous medium” as used herein refers to any composition or medium comprising water in the free or liquid state, and not bound in a dry medium such as water of crystallization. In the context of the systems receiving the photoacoustic probes of the present disclosure, an aqueous medium can be, but is not limited to, a biological cell, a biological tissue or organ, or a biological fluid, including such as blood, interstitial fluid surrounding a tissue in an animal or human body, and the like, or a pharmaceutically acceptable water-based solvent.

The term “cancer” as used herein shall be given its ordinary meaning and is a general term for diseases in which abnormal cells divide without control. Cancer cells can invade nearby tissues and can spread through the bloodstream and lymphatic system to other parts of the body. There are several main types of cancer, for example, carcinoma is a cancer that begins in the skin or in tissues that line or cover internal organs. Sarcoma is a cancer that begins in the bone, cartilage, fat, muscle, blood vessels, or other connective or supportive tissue. Leukemia is cancer that starts in blood-forming tissue such as bone marrow, and causes large numbers of abnormal blood cells to be produced and enter the bloodstream. Lymphoma is cancer that begins in the cells of the immune system. The probes and methods of the disclosure are especially advantageous for detecting cancer cells and tumors localized to a specific site in an animal or human, although it is contemplated that the systems may be useful to detect circulating cells.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous), or malignant (cancerous). Different types of solid tumors are named for the type of cells that form them.

The term “cellulose” refers to a polysaccharide containing at least two D-glucose units. Cellulose is an organic compound with the formula (C₆H₁₀O₅)ₙ, a polysaccharide consisting of a linear chain of several hundred to over ten thousand β(1→4) linked D-glucose units. Cellulose may come from many sources including plants.

The term “cellulase” refers to an enzyme known to reduce cellulose into smaller sugars such as, but not limited to, glucose.

The term “cotton linter” as used herein refers to fine, silky cotton fibers that adhere to the seeds of the cotton plant after ginning. These curly fibers typically are less than 3 mm long. The term also may apply to the longer textile fiber staple lint as well as the shorter fuzzy fibers from some upland cotton species. Linters are traditionally used in the manufacture of paper and as a raw material in the manufacture of cellulose. Cotton linter may also be known as “cotton wool,” or “absorbent cotton.”

The term “delivery” as used herein can refer to contacting an isolated cell, a population of cultured cells originating from a subject animal or human, a tissue within or isolated, in whole or in part, from an animal or human subject, or administration of the cellulose nanoparticles of the disclosure to an animal or human subject.

The term “detectable” refers to the ability to detect a signal over the background signal. The detectable signal is defined as an amount sufficient to yield an acceptable image using equipment that is available for pre-clinical use. A detectable signal maybe generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

The term “detecting” refers to detecting a signal generated by one or more photoacoustic probes. It should be noted that reference to detecting a signal from a photoacoustic probe also includes detecting a signal from a plurality or concentration of photoacoustic probes. In some embodiments, a signal may only be detected that is produced by a plurality of photoacoustic probes. Additional details regarding detecting signals (e.g., acoustic signals) are described below.

The term “dispose” as used herein refers to the permanent or temporary attachment of matter to a supporting material.

The term “dye compound” as used herein refers to a fluorescent molecule, i.e., one that emits electromagnetic radiation, especially of visible light, when stimulated by the absorption of incident radiation. The term includes, but is not limited to, fluorescein, a xanthene dye having an absorption maximum at 495 nanometers. A related fluorophore is Oregon Green, a fluorinated derivative of fluorescein. The term further includes bora-diaza-indecene, rhodamines, and cyanine dyes. A “rhodamine” is a class of dyes based on the rhodamine ring structure. Rhodamines include (among others): Tetramethylrhodamine. (TAMRA) and carboxy tetramethyl-rhodamine. Rhodamines are established as natural supplements to fluorescein based fluorophores, which offer longer wavelength emission maxima and thus open opportunities for multicolor labeling or staining. The term is further meant to include “sulfonated rhodamine,” a series of fluorophores known as ALEXA.RTM FLUOR dyes (Molecular Probes, Inc.). These sulfonated rhodamine derivatives exhibit higher quantum yields for more intense fluorescence emission than spectrally similar probes, and have enhanced photostability, absorption spectra matched to common laser lines, pH insensitivity, and a high degree of water solubility. “Dye compound” may also refer to any molecule known to quench fluorescence including QXL dyes, black holes quenchers and the like.

“Cyanines” are a family of cyanine dyes, Cy2, Cy3, Cy5, Cy7, and their derivatives, based on the partially saturated indole nitrogen heterocyclic nucleus with two aromatic units being connected via a polyalkene bridge of varying carbon number. These probes exhibit fluorescence excitation and emission profiles that are similar to many of the traditional dyes, such as fluorescein and tetramethylrhodamine, but with enhanced water solubility, photostability, and higher quantum yields. The excitation wavelengths of the Cy series of synthetic dyes are tuned specifically for use with common laser and arc-discharge sources, and the fluorescence emission can be detected with traditional filter combinations. Cyanine dyes are available as reactive dyes or fluorophores coupled to a wide variety of secondary antibodies, dextrin, streptavidin, and egg-white avidin. The cyanine dyes generally have broader absorption spectral regions than members of the Alexa Fluor family.

The term “illuminating” as used herein refers to the application of a light source such as, but not limited to, near-infrared (NIR), visible light, including laser light capable of exciting dyes and nanoparticle cores of the embodiments of the probes herein disclosed.

The term “in vivo imaging” as used herein refers to methods or processes in which the structural, functional, or physiological state of a living being is examinable without the need for a life-ending sacrifice.

The terms “linker” or “spacer” as used herein refer to a molecule or group of molecules that connects two molecules, such as, for example, a target-specific binding ligand and nanoparticle of the disclosure, and serves to place the two molecules in a preferred configuration.

The term “kit” as used refers to a packaged set of related components, typically one or more compounds or compositions, and typically includes containers for the components of the kit, instructions for their use according to the methods of the present disclosure, advertising, trademarks, etc.

The term “nanoparticle” refers to a material with dimensions below 1000 nm. These dimensions may be extended in any of three axes (x, y, and z). Nanoparticles may be made naturally or synthetically. Nanoparticles may consist of multiple components including core, shell, and coat. The ratio of core, shell, and coat may differ between different batches of nanoparticles. The cellulose nanoparticles of the disclosure can be, but are not limited to, anisotropic forms (the property of being directionally dependent, as opposed to isotropy, which implies identical properties in all directions)

The term “non-invasive in vivo imaging” as used herein refers to methods or processes in which the structural, functional, or physiological state of a being is examinable by remote physical probing without the need for breaching. “Non-invasive in vivo imaging as used herein refers to administering a detectable photoacoustic probe to a living subject, and then generating and/or detecting a signal from the probe using a detector that has not breached the physical integrity of the outer (skin) or inner (accessible orifices) surfaces of the body.

The term “optical energy” as used herein refers to electromagnetic radiation between the wavelengths of about 350 nm to about 950 nm and which can be absorbed by the dyes or cellulose-based nanoparticles of the embodiments of the photoacoustic probes of the disclosure. The term “optical energy” may be construed to include laser light energy or non-laser energy.

The term “particle” or “nanoparticle” as used herein refers to a contrast agent with dimensions of about 1 and 5000 nm.

The term “phantom” as used herein refers to a specimen created for the purposes of measuring acoustic detectable signal or other signal. These phantoms may be made of agar and other material.

The term “pharmaceutically acceptable carrier” as used herein refers to a diluent, excipient, or vehicle with which a probe of the disclosure can be administered and which is approved by a regulatory agency of the Federal or a state government or listed in the U.S. Pharmacopeia or other generally recognized pharmacopeia for use in animals. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for delivering the contrast agents of the disclosure to an animal or human subject.

The term “photoacoustic detectable signal” as used herein refers to a signal derived the contrasting agent absorbing light energy and converting it to thermal energy that generates the photoacoustic signal. The photoacoustic detectable signal is detectable and distinguishable from other background photoacoustic signals that are generated from the subject or sample. In other words, there is a measurable and statistically significant difference (e.g., a statistically significant difference is enough of a difference to distinguish among the photoacoustic detectable signal and the background, such as about 0.1%, 1%, 3%, 5%, 10%, 15%, 20%, 25%, 30%, or 40% or more difference between the photoacoustic detectable signal and the background) between photoacoustic detectable signal and the background. Standards and/or calibration curves can be used to determine the relative intensity of the photoacoustic detectable signal and/or the background.

The term “photoacoustic imaging” as used herein refers to signal generation caused by a light pulse, absorption, and expansion of a contrast agent, followed by acoustic detection, where the contrasting agent absorbs the light energy and converts it to thermal energy that generates the photoacoustic signal. “photoacoustic imaging” further refers to converting the detectable signal into a data form that may then be transformed into an image of the signal within the cell/s, tissue/s, or living animal or human, said image being visible and interpretable by an operator.

The term “optical detectable signal” (e.g., a fluorescent signal) as used herein refers to a signal derived from a particle that absorbs light and converts absorbed energy into optical energy of a different wavelength. The optical detectable signal is detectable and distinguishable from other background optical signals that are generated from the subject or sample. In other words, there is a measurable and statistically significant difference (e.g., a statistically significant difference is enough of a difference to distinguish among the optical detectable signal and the background, such as about 0.1%, 1%, 3%, 5%, 10%, 15%, 20%, 25%, 30%, or 40% or more difference between the ultrasound detectable signal and the background) between ultrasound detectable signal and the background. Standards and/or calibration curves can be used to determine the relative intensity of the ultrasound detectable signal and/or the background.

The term “sample” as used herein can refer to a tissue sample, cell sample, a fluid sample, and the like. The sample may be taken from a host and may also refer to an isolated or cultured population of cells. The tissue sample, which can be in a living animal or human subject, or is isolated therefrom, can include brain, hair (including roots), buccal swabs, blood, saliva, semen, muscle, or from any internal organs, or cancer, precancerous, or tumor cells associated with any one of these. The fluid may be, but is not limited to, urine, blood, ascites, pleural fluid, spinal fluid, and the like. The body tissue can include, but is not limited to, brain, skin, muscle, endometrial, uterine, and cervical tissue or cancer, precancerous, or tumor cells associated with any one of these.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscides, Perissodactyla, Artiodactyla, Rodentia, and Lagomorphs. In a particular embodiment, the mammal is a human. In the context of the disclosure, the term “subject” can refer to an individual who will receive or who has received a photoacoustic nanoparticle probe according to the disclosure that is administered with the intention of obtaining photoacoustic image therefrom and, optionally, for the treatment of a targeted pathological condition such as a cancer. In certain aspects, a subject may be a healthy subject. Typical subjects for treatment include persons afflicted with or suspected of having or being pre-disposed to a disease disclosed herein, or persons susceptible to, suffering from or that have suffered a disease disclosed herein.

The term “target” as used herein refers to a peptide, cell, tissue, tumor, etc., for which it is desired to detect. The target may be exposed totally or in part at a cell surface or cell, the cell being isolated from an animal host, a cultured cell or a cell or population of cells in a tissue of an animal. Targets may be present in the vasculature, cell surface, in any region inside the cell, or in the matrix between cells.

The term “therapeutically effective amount” as used herein refers to that amount of an embodiment of the agent being administered that will relieve to some extent one or more of the symptoms of the disease or condition being treated, and/or that amount that will prevent, to some extent, one or more of the symptoms of the disease or condition that the subject being treated has or is at risk of developing.

The term “thermal energy” as used herein refers to electromagnetic radiation of wavelengths between about 700 nm and about 1000 nm and which can increase the temperature of a medium exposed to such radiation.

The terms “treat,” “treatment,” “treating” and the like, as used herein refer to acting upon a disease, condition, or disorder with an agent to affect the disease, condition, or disorder by improving or altering it. The improvement or alteration may include an improvement in symptoms or an alteration in the physiologic pathways associated with the disease, condition, or disorder. “Treatment,” as used herein, covers one or more treatments of a disease, condition, or disorder in a subject (e.g., a mammal, typically a human or non-human animal of veterinary interest), and includes: (a) reducing the risk of occurrence of the disease in a subject determined to be predisposed to the disease, condition, or disorder but not yet diagnosed (b) impeding the development of the disease, condition, or disorder, and/or (c) relieving the disease, condition, or disorder, e.g., causing regression of the disease, condition, or disorder and/or relieving one or more disease, condition, or disorder symptoms.

### Description

It is within the scope of the present disclosure to include cellulose photoacoustic probes, methods of making photoacoustic probes, methods of imaging, and the like. Embodiments of the photoacoustic probes are able to detect one or more targets (e.g., cells, tissue, tumors, chemicals, enzymes, and the like) by detecting the generation of an acoustic signal. Most advantageously, the cellulose nanoparticles of the disclosure are biodegradable by the action of cellulases. Accordingly, clearance of the nanoparticles from a subject cell or tissue is significantly enhanced, and results in improved contrast of a photoacoustic-generated image by reducing background signals.

It is contemplated that embodiments of the cellulose-based photoacoustic probes of the disclosure may further include a dye or a plurality of dyes bound thereto. Relative to cellulose-based particles not including a dye, embodiments of the present disclosure may then have increased absorption by a factor of 5 or more, 10 or more, 15 or more, 20 or more, 30 or more, 40 or more, 50 or more, 60 or more, or 70 or more. In addition, embodiments of the present disclosure may have an increase in the photoacoustic signal by a factor of 5 or more, 10 or more, 15 or more, 17 or more, or 20 or more, relative to cellulose-based photoacoustic probes not including the dye(s). Thus, embodiments of the present disclosure can be advantageous over bare cellulose-based photoacoustic probes.

It is also contemplated that a targeting moiety can be attached (e.g., directly, or indirectly by means of a linker) to the photoacoustic cellulose probes of the disclosure so that an image of a specific target can be correlated with the acoustic signal. In other words, an image of the target can be created using the acoustic signal from the photoacoustic probe.

Embodiments of the photoacoustic probes of the disclosure are useful for providing high acoustic contrast for imaging. In this regard, the photoacoustic probes can be used for imaging anatomical and/or physiological events in a host. Embodiments of the present disclosure enable the imaging of anatomical and/or physiological and/or molecular events in vitro or in vivo using photoacoustic techniques and methods. The image acquired using the photoacoustic probes can be used to illustrate the concentration and/or location of the photoacoustic probes. In embodiments where the photoacoustic probes is labeled with a targeting moiety that has an affinity for a target (e.g., tumor), the image acquired can be correlated with the location and/or dimensions of the target. The contrast of the acquired image may be enhanced by degradation of the cellulose particles and their clearance from a subject. Thus, the degradation products are typically sugar moieties that may be readily metabolized by cellular-based pathways or excreted by renal clearance. This degradation results in reductions in the background signal, allowing a more certain detection of an accumulation of the probes in a target cell or tissue of the animal or human subject.

The cellulose-based nanoparticles of the disclosure and dye compounds that may be attached thereto can combine to absorb optical energy and convert it to thermal energy to produce a detectable acoustic signal. The dye molecules may be non-covalently bound to the cellulose-based nanoparticles such as, but not limited to, pi-pi stacking interactions. An advantage of the pi-pi stacking is ultra-high loading and increased quenching leading to increased photoacoustic signal. The dye molecules may also be bound to the cellulose-based nanoparticles via covalent conjugation.

The cellulose-based nanoparticles of the present disclosure may have a plurality of dye molecules bound thereto and a targeting moiety attached (e.g., directly or indirectly) to the nanoparticles. The targeting moiety can be used to direct the photoacoustic probe to a target. Detection of the acoustic signal can then be correlated with an image of the target (e.g., a tumor) and used to determine the location and/or dimensions of the target.

In general, suitable targeting moieties for attachment to the probes of the disclosure can include, but are not limited to, polypeptides (e.g., proteins such as, but not limited to, antibodies (monoclonal or polyclonal)), nucleic acids (both monomeric and oligomeric), polysaccharides, sugars, fatty acids, steroids, purines, pyrimidines, ligands, or combinations thereof. The targeting moiety can have an affinity for one or more targets. In general, the target can include, but is not limited to, a cell type, a cell surface, extracellular space, intracellular space, a tissue type, a tissue surface, the vascular, a polypeptide, a nucleic acid, a polysaccharide, a sugar, a fatty acid, a steroid, a purine, a pyrimidine, a hapten, a ligand, and the like, related to a condition, disease, or related biological event or other chemical, biochemical, and/or biological event of the sample or host. The targeting moiety can be selected based on the target selected and the environment the target is in and/or conditions that the target is subject to.

A non-specific targeting moiety can be selected to do one or more of the following: enter a cell or a cell type, enter the vasculature, enter extracellular space, enter intracellular space, have an affinity for a cell surface, diffuse through a cell membrane, react with a non-specified moiety on the cell membrane, enter tumors due to leaky vasculature, and the like. The non-specific targeting moiety can include a chemical, biochemical, or biological entity that facilitates the uptake of the photoacoustic probe into a cell. The non-specific targeting moiety can include, but is not limited to, cell penetrating peptides, polyamino acid chains, small molecules, and peptide mimics.

The targeting moiety can be linked, directly or indirectly, to the cellulose-based nanoparticles of the disclosure in a manner described above using a stable physical, biological, biochemical, and/or chemical association. In general, the targeting moiety can be independently linked via chemical bonding (e.g., covalently or ionically), biological interaction, biochemical interaction, and/or otherwise associated with the cellulose-based nanoparticles of the disclosure in a manner described above. The targeting moiety can be independently linked using a link such as, but not limited to, a covalent link, a non-covalent link, an ionic link, a chelated link, as well as being linked through interactions such as, but not limited to, hydrophobic interactions, hydrophilic interactions, charge-charge interactions, π-stacking interactions, combinations thereof, and like interactions.

A targeting moiety may be attached to the cellulose-based nanoparticles via a linker such as a polyethylene glycol polymer, dextran, or the like. The PEG can be a linear PEG, a multi-arm PEG, a branched PEG, and combinations thereof. The molecular weight of the PEG can be about 1 kDa to 100 kDa, about 1 kDa to 50 kDa, about 1 kDa to 40 kDa, about 1 kDa to 30 kDa, about 1 kDa to 20 kDa, about 1 kDa to 12 kDa, about 1 kDa to 10 kDa, and about 1 kDa to 8 kDa.

In some embodiments of the cellulose-based nanoparticles of the disclosure, the amount of PEG polymer bound to a CNP can be about 20 to 500 PEG polymer compounds per CNP. In another embodiment, the amount of PEG polymer bound to a CNP is about 50 to 250 PEG polymer compounds per CNP. In another embodiment, the amount of PEG polymer bound to a CNP is about 100 to 200 PEG polymer compounds per CNP. The amount of PEG per CNP may be higher than 250 PEG polymer compounds per CNP. The molecular weight of this PEG may be between 3000 to about 12000 kDa. Short chain (n=4 PEG) hetero- or homo-bifunctional cross-linkers may also be used.

It should be noted that other compounds can be used to link PEG to the CNP. For example, but not intended to be limiting, PEGylated poly-pyrene can be used. PEGylated fatty acid with a lipid chain length greater than about 20 can also be used. Other covalent reactions can be used to attach the PEG to the cellulose-based nanoparticles and functionalization chemistry can be used to accomplish this end. It should also be noted that non-covalent functionalization can be used to link the PEG to the cellulose-based nanoparticles.

The present disclosure further encompasses methods of generating images of targeted cells or tissues by administering a population of the cellulose nanoparticle probes of the disclosure to an animal or human subject, exposing the subject to an irradiating illumination of a wavelength exciting the nanoparticles to emit photoacoustic energy detectable by sensors external to the subject. The detectable signal from the subject may then be converted to a detectable and visual image of the concentration of the nanoparticle probes in the subject. It is further contemplated that the methods of imaging according to the disclosure may further include the step(s) of allowing a cellulase to degrade nanoparticles to a level such that undesirable background emitted signals are substantially reduced, thereby increasing the contrast and quality of the generated image. It is further contemplated that if a subject animal, or cells or tissues thereof does not include an endogenous source of at least one enzyme capable of degrading the cellulose molecules of the nanoparticles, a pharmaceutically acceptable composition comprising an effective dose of such an enzyme, for example a cellulase, may be administered simultaneously with, before or after the administration of the nanoparticles.

This disclosure further encompasses kits that include, but are not limited to, photoacoustic probes and directions (written instructions for their use). The components listed above can be tailored to the particular disease, biological event, or the like, being studied, imaged, and/or treated (e.g., cancer, cancerous, or precancerous cells). The kit can further include appropriate buffers and reagents known in the art for administering various combinations of the components listed above to the host cell or host organism.

The acoustic energy can be detected and quantified in real time using an appropriate detection system. The acoustic signal can be produced by one or more photoacoustic probes of the disclosure. One possible system is described in the following references: J. Biomedical Optics (2006) 11, p024015; Optics Letts. 30: 507-509, each of which are included herein by reference. In an embodiment, the acoustic energy detection system can includes a 5 MHz focused transducer (25.5 mm focal length, 4 MHz bandwidth, F number of 2.0, depth of focus of 6.5 mm, lateral resolution of 600 μm, and axial resolution of 380 μm. A309S-SU-F-24.5-MM-PTF, Panametrics), which can be used to acquire both pulse-echo and photoacoustic images. In addition, high resolution ultrasound images can also be simultaneously acquired using a 25 MHz focused transducer (27 mm focal length, 12 MHz bandwidth, F number of 4.2, depth of focus of 7.5 mm, lateral resolution of 250 μm, and axial resolution of 124 μm. V324-SU-25.5-MM, Panametrics). Other detection strategies including capacitive micromachined ultrasonic transducers (CMUT) arrays can also be used to detect the acoustic signal.

The cellulose nanoparticles of the disclosure can include NIR dyes attached thereto. The NIR dyes can include, but are not limited to, BODIPY.RTM fluorophores (Molecular Probes) (e.g., 4,4-difluoro-4-bora-3a,4a-diaza-s-indacene (and derivatives thereof), which can be modified to alter the wavelength (BODIPY® substitutes for the fluorescein, rhodamine 6G, tetramethylrhodamine and Texas Red fluorophores are BODIPY.RTM FL, BODIPY.RTM R6G, BODIPY.RTM TMR and BODIPY.RTM TR, respectively)), 1H, 5H, 11H, 15H-Xantheno[2,3,4-ij:5,6,7-i′j′]diquinolizin-18-ium, 9-[2(or 4)-(chlorosulfonyl)-4(or 2)-sulfophenyl]-2,3,6,7,12,13,16,17-octahydro-, inner salt (molecular formula: C31H29CIN2O6S2) (and derivatives thereof) (Texas Red), Xanthylium, 3,6-diamino-9-(2-(methoxycarbonyl) phenyl, chloride (C₂₁H₁₇CIN₂O₃) (and derivatives thereof) (NIR Rhodamine dye), and cyanine dyes (and derivatives thereof), where derivatives of each can be used to modify the wavelength. In particular, the fluorescent compound can include, but is not limited to, BODIPY.RTM dye series (e.g., BODIPY.RTM FL-X, BODIPY.RTM R6G-X, BODIPY.RTM TMR-X, BODIPY.RTM TR-X, BODIPY.RTM 630/650-X, and BODIPY.RTM 650/665-X (Molecular Probes, Inc. Eugene, Oreg., USA)), NIR Rhodamine dyes, NIR ALEXA.RTM dyes (e.g., ALEXA.RTM Fluor 350, ALEXA.RTM Fluor 405, ALEXA.RTM Fluor 430, ALEXA.RTM Fluor 488, ALEXA.RTM Fluor 500 (Molecular Probes, Inc. Eugene, Oreg., USA)), Texas Red, or cyanine dyes (e.g., Cy5.5 Cy3, Cy5), and Li-Cor IRDye™ products.

A linker can be a compound or polymer that includes one or more functional groups to attach one or more targeting moiety. The linker can include functional groups such as, but not limited to, amines, carboxylic acids, hydroxyls, thiols, and combinations thereof. The linker can include compounds such as, but not limited to, diethylene triamine pentaacetic acid (DTPA), ethylenediaminetetraacetic acid (EDTA), 3,4-dihydroxyphenylalanine (DOPA), ethylene glycol tetraacetic acid (EGTA), nitrilotriacetic acid (NTA), and combinations thereof.

It should be noted that an agent other than targeting agents or dyes could be included in the photoacoustic probe. The agent can be linked to one of the targeting moiety or the non-fluorescent absorber compound directly or indirectly. The agent can include, but is not limited to, polypeptides (e.g., protein such as, but not limited to, an antibody (monoclonal or polyclonal)), nucleic acids (both monomeric and oligomeric), polysaccharides, sugars, fatty acids, steroids, purines, pyrimidines, drugs (e.g., small compound drugs), ligands, photosensitizers, or combinations thereof.

In addition, the suitable agents can also include, but are not limited to, a drug, a therapeutic agent, radiological agent, photosensitizers, a small molecule drug, and combinations thereof, that can be used to treat the target molecule and/or the associated disease and condition of interest. The drug, therapeutic agent, or radiological agent can be selected based on the intended treatment as well as the condition and/or disease to be treated. In some embodiments, the photoacoustic probe can include two or more agents used to treat a condition and/or disease. In addition, the detection of the photoacoustic probe can be used to ensure the delivery of the agent or drug to its intended destination as well as the quantity of agent or drug delivered to the destination.

In particular, the photoacoustic probes of the disclosure can be used in in vivo diagnostic and/or therapeutic applications such as, but not limited to, targeting diseases and/or conditions and/or imaging diseases and/or conditions. For example, one or more embodiments of the photoacoustic probes can be used to identify the type of disease or condition, identify the presence of one or more compounds associated with the disease or condition, locate the proximal locations of the disease or condition, and/or deliver agents (e.g., drugs) to the diseased cells (e.g., cancer cells, tumors, and the like) in living animals.

The photoacoustic probes of the disclosure can further comprise one or more agents to treat the cancerous cells, precancerous cells, cancer, or tumors. Upon measuring the acoustic signal generated from the administered photoacoustic cellulose probe, one can determine if the photoacoustic probe has coordinated with the cancerous cells, precancerous cells, cancer, or tumors. Embodiments of the photoacoustic probe can aid in visualizing the response of the cancerous cells, precancerous cells, cancer, or tumors to the agent. Accordingly, the photoacoustic probe may also contain a therapeutic agent (small molecule, protein, etc.) and release it after administration to a living subject. This release may or may not be dependent upon the heat generated during photoacoustic imaging.

It is further contemplated that the degradable nanoparticle probes of the disclosure can be useful to label a cell or tissue ex vivo before delivery to an animal or human subject. Such implanted labeled cells may then be tracked to determine their movement and locations within the recipient subject.

The cellulose-based degradable nanoparticles of the disclosure may be generated from a variety of cellulose-based materials including, but not limited to, cotton linter or a purified form of cellulose such as, but not limited to, SIGMACELL.RTM. A particularly advantageous and readily available source of cellulose is cotton linter that is used in paper making. It has been determined that the physical properties of the CNPs may be adjusted or selected based on the type of cellulose used.

During the exothermic synthesis of the CNPs of the disclosure, as described in Example 2, an opaque white suspension of cellulose that readily precipitates was converted to a dark brown colloidal suspension. After purification, the CNPs derived from such as cotton linters could be concentrated by centrifugation and were colloidally stable, with no sign of degradation over at least six months. The rate of addition of acidic components to the aqueous suspension of cellulose has an influence on the properties of the cellulose nanoparticles. A slow addition of acid produced particles unable to generate a useful photoacoustic signal. A too rapid addition resulted in rapid heating and the likely formation of charcoal as well as a dangerous exothermic schema. The synthesis removes D-glucose monomers with lower coordination and leaves the highly coordinated, more crystalline cellulose intact.

The linter-derived CNPs of the disclosure were suspended in water to an optical density (OD) of approximately 0.2 at 700 nm and studied with TEM, absorbance spectroscopy, and DLS.

It was found that the inclusion of uranyl acetate for increased contrast was optional and the images shown in FIGS. 1A-1C were obtained in the absence of such positive staining. TEM images were analyzed with ImageJ to determine the mean diameter of representative nanoparticles, which was about 132±46 nm. The DLS data, as shown in FIG. 1D, in 50:50 water:PBS presents particles with a mean size between about 160 to about 200 nm, a polydispersity index of about 0.138, and a zeta potential of between about −0.1 to about 0.1 mV.

CNPs formed using SIGMACELL.RTM as the cellulose source had a hydrodynamic radius greater than about 1000 nm and were not colloidally stable. Accordingly, although the SIGMACELL-based CNP's were considered useful, the most advantageous embodiments of the CNPs of the disclosure were formed from cotton linter with a typical weight concentration of about 2.4 mg/mL. Using the value of 1.5 g/cm³ as the density of cellulose, the molecular weight for these 3.8×10⁶ nm³ CNPs was calculated to be about 3.4×10⁹ g/mol, or 0.70 nM in the 2.4 mg/mL batch. Absorbance spectroscopy data, as shown in FIG. 1E, showed Rayleigh scatter and that the CNPs had their most intense interaction with light in the visible range of the spectrum. This is in contrast to gold nanorods at 0.34 nM that show a typical near infra-red absorbance peak. Evaporation can be used to determine the concentration of the CNPs in units of mass per volume.

The capacity of the CNPs of the disclosure to generate a photoacoustic signal was characterized, initially by optimizing the imaging wavelength. Because in vivo imaging was the ultimate goal, a 100 μL bolus of 0.6 mg/mL CNPs in 50% matrigel was subcutaneously injected into the rear limb of a nude mouse. The PA-intensity spectrum of the injected bolus was collected with a LAZR scanner and plotted, as shown in FIG. 2A. Also shown is the spectrum of normal tissue not treated with CNPs. The data show that an optimal signal occurs at about 700 nm and this wavelength was used for all subsequent experiments.

Decreasing concentrations of CNPs were placed in the dimple of the tomographic photoacoustic scanning tray and imaged with 700 nm excitation. The reconstructed images are shown, for example, in FIG. 2B and show the concentration-dependent nature of the PA signal. Absolute quantitation is shown in FIG. 2C together with data from gold nanoparticles (GNRs) having dimensions of 40.0±4.4 and 13.2±1.8 nm, longitudinally and axially, respectively.

The peak absorbance of the GNRs was 700 nm, which was also used as the excitation wavelength for the comparison studies. For the CNPs, there was discrimination between the 0.00 nM and 0.01 nM data points (p <0.01) and the calculated LOD was 0.006 nM (0.02 mg/mL). Experiments at isomolar concentrations of CNPs and GNRs showed that the CNPs have an increased signal relative to GNRs: 3.0-fold at 0.15 nM, 2.0-fold at 0.35 nM, and 1.7-fold at 0.70 nM. Regression analysis between the molar concentration and PA signal indicates a R² value greater than 0.99 for both the GNRs and CNPs. The slope of the CNP curve was 1.6-fold greater than the GNR curve.

The CNPs could be also separated into at least two different fractions with centrifugation at 20,000×g for 30 min. Particles remaining in the supernatant had a mean size of about 65 nm by DLS, whereas the re-suspended sediment had a mean size of about 186 nm. When the two fractions were brought to the same optical density, the photoacoustic signal in the 65 nm fraction was 1.5-fold greater than that of the larger fraction. In further embodiments, fractionation may separate different sizes of CNPs for different applications.

In vitro tissue culture experiments and small animal models were used to evaluate in the imaging agent's toxicity. The CNPs were first tested with 10⁴ OV2008 cells plated in each well of a 96-well plate. Increasing concentrations of CNPs were added to the growth media and incubated overnight. Analysis with the PRESTOBLUS.RTM assay indicated a small, but statistically significant (p <0.05) decrease in metabolic activity at concentrations above 0.31 mg/mL of CNPs, as shown in FIG. 3A.

CNPs were also injected via tail vein into mice (n=3), and whole blood was collected retro-orbitally 24 h later for serum chemistry (Na, K, Cl, CO₂, and anion gap) and liver function tests (aspartate transaminase (AST), alanine transaminase (ALT), alkaline phosphatase (AlkPhos), gamma-glutamyltransferase (GGT), and total bilirubin). Three CNP concentrations were studied: 2.4 mg/mL, 1.2 mg/mL, and 0.1 mg/mL. Samples from control animals with no injection of an imaging agent were also tested.

The cohorts receiving 1.2 mg/mL and below had no obvious signs of toxicity including modified behavior, posture, or activity. Serum liver function tests and electrolytes indicated no statistically significant changes for the 0.1 mg/mL cohort (p >0.05). The 1.2 mg/mL animals had statistically significant (p <0.05) changes to alkaline phosphatase, sodium, CO₂, and the calculated anion gap. Total bilirubin was not detectable in any of the samples and GGT was only detectable in one of the control animals.

A glucose assay and TEM imaging to evaluate the biodegradation of the CNPs in the presence of cellulase were also performed. In the former, cellulose degrades into component glucose, which can be readily measured by a hexokinase-based glucose assay.

The activity of the cellulase was validated, as shown in FIG. 4B. Controls included cellulose only (Negative control; Cell-Enz.) and cellulose with cellulase (Positive control; Cell+Enz.). The results indicated that free glucose was liberated from the cellulose by cellulase, but not from the cellulose in the absence of this enzyme. This “cellulase-free” experiment was repeated for all of the subsequent CNP experiments shown in FIG. 4C. The A₃₄₀ for these samples (due to solvent) was subtracted from the “cellulase-positive” experiments. The A₃₄₀ for a “cellulase only” sample from each experiment was corrected for absorbance from the enzyme's protein structure.

The cotton linter-derived CNPs of the disclosure were preferred due to their smaller size and colloidal stability. However, the biodegradation of both linter and Sigmacell-derived CNPs was compared. These samples were studied at 1 mg/mL along with naïve cellulose. The results show rapid biodegradation of cellulose and of both CNPs types. The linter CNP reached 50% of maximum values between 30-60 min, while the Sigmacell-derived CNPs achieve this value more slowly, between 60-120 min. While the starting concentrations were the same, more glucose was released from the CNPs than from the cellulose standards: 3.8-fold from Sigmacell-derived CNPs and 3.1-fold for the linter CNPs.

DLS and TEM studies were performed on the linter CNPs before and after enzyme treatment. As a negative control, TEM was also performed on CNPs subjected to 37° C. heating in acetic acid solution for 1 h, but without cellulase. The TEM images showed a marked impact of cellulase treatment on the CNPs. Naïve CNPs, as shown in FIG. 4D, showed a morphology typical of that shown in FIG. 1A, while CNPs treated with cellulase for one hour showed small fragments near the main body of the nanoparticle (arrowed, FIG. 4E). ROI analysis on 25 of these fragments from multiple fields of view determined the size to be about 12.0 nm±3.2 nm. Further quantification across at least 10 different fields of view indicated that each field in the enzyme-treated sample contained about 23±16 fragments versus about 4±4 fragments in the control sample, a five-fold increase that was significant at p=0.003. Less than one such 12 nm fragment was seen per field-of-view in the pre-treatment CNP samples. There was no difference in DLS peak for any of the three samples.

The in vivo imaging potential of CNPs was also studied. Nude mice bearing subcutaneous xenograft tumors from the OV2008 cell line of between about 500 mm³ to about 1000 mm³were fitted with a tail vein catheter and the tumor placed in the imaging dimple of the Endra PA imaging system. Scans were collected prior to injection of contrast and 1, 15, 30, 45, and 60 min post-injection. Because there was a wide variety in the baseline photoacoustic signal between animals, each mouse was considered to be its own control, and the PA signal for each scan was plotted relative to the pre-injection signal, as shown in FIG. 5A. Three different concentrations were used: 1.2 mg/mL, 2.4 mg/mL, and 4.0 mg/mL with a constant injection volume of 200 μL, expressed as 0.024 mg, 0.048 mg, and 0.8 mg (0.07 nmol, 0.14 nmol, and 0.23 nmol, respectively). A group without injected contrast was also studied. Three mice were imaged at each point except for the highest concentration at which only 2 mice were studied.

Although only the 2.4 mg/mL cohort is shown in FIG. 5A, the remaining data sets were similar with a maximum PA signal occurring between 15-30 min post injection and with a T½ of between 7-15 min. As shown in FIG. 5B, the maximum fold increase above baseline was plotted for the different concentration regimes. The relationship between injected concentration and signal increase was linear at R²>0.98. All injected concentrations were statistically (p <0.05) elevated relative to the control group. A washout period within 30 min after peak intensity was seen; however, the tumor PA signal did not return to the baseline level during the imaging session and decreased at most by about 20%. After data collection, the imaging data was rendered as maximum intensity projection images along the axial plane, as shown in FIG. 6A. These images showed an increase in the tumor signal when the pre-injection results (FIGS. 6B-6E) are compared to the post-injection images (FIGS. 6Bi-6Ei).

Accordingly, the disclosure provides a cellulose-based nanoparticle imaging agent suitable for photoacoustic imaging with the capacity to biodegrade. Although cellulose nanocrystals and microcrystals are primarily described as optical reflection tools (de Souza Lima & Borsali (2004) Macromol. Rapid Comms. 25: 771-787; Capadona et al., (2009) Biomacromols. 10: 712-716; Samir et al., (2005) Biomacromols. 6: 612-626), they have also shown interaction with the infrared spectrum that may be responsible for the photoacoustic signal described here via thermal expansion (Akerholm et al., (2004) Carbohydrate Res. 339: 569-578). The CNPs of the disclosure produce 2-3 times as much PA signal as GNRs on a per-particle basis at 700 nm.

The imaging agents of the disclosure had a sharp response curve, and doses presented to mice at or above 2.4 mg/mL showed toxicity. However, at 1.2 mg/mL there was only very slight modification of liver function and electrolytes relative to control animals with changes still within the normal reference range. The most dramatically changed enzyme, alkaline phosphatase, actually decreased whereas liver toxicity would typically show this as an increase (see FIG. 3B). Importantly, this same 1.2 mg/mL concentration was still effective at producing a significant increase in tumor PA signal, as shown in FIGS. 5A, 5B, and 6 (Panels A-Ei).

This concentration was further validated by the ex vivo experiments. The 200 μL of 1.2 mg/mL CNPs would have a working concentration in vivo of 0.12 mg/mL for a typical mouse with 2 mL of total blood volume. This value is below the 0.31 mg/mL value shown to induce dysregulated metabolisms by cell culture experiments, as shown in FIG. 3A.

FIG. 4 shows both that chemical and imaging data support the hypothesis that CNPs can biodegrade in the presence of cellulase. While free glucose is likely to be used metabolically, the smaller fragments are approximately the size known to clear renally (Choi et al., (2007) Nature Biotech. 25: 1165-1170). At the same 1 mg/mL starting concentration, temperature, and enzyme activities, there was 3.1-fold more glucose with the linter CNP and 3.9-fold more glucose produced with the Sigmacell-derived CNP, consistent with the acidic cleavage of cellulose during synthesis with a potential attendant increase in enzymatic reaction sites on the newly formed CNPs.

Unlike mice, humans do not produce cellulase. It is contemplated, therefore, that for use in human patients it would be advantageous to provide a secondary administration of an effective dose of a heterogenous cellulase activity. The probes of the disclosure, therefore, can be advantageously used as a “smart probe” that biodegrades after injection of the secondary cellulase-based treatment. It is further contemplated that there could be embodiments of the cellulose-based nanoparticles of the disclosure that incorporate modified sugar backbones that retain the optical and photoacoustic properties, but with alternative or efficient routes of clearance from the body due, for example, to greater susceptibility to cellulase degradation.

In humans, imaging at 700 nm can be confounded by hemoglobin and other molecular species in tissue. Nevertheless, registration and comparison of the pre- and post-injection images can highlight a CNP-specific signal and this value is still lower than that determined for GNRs with identical cell line and imaging equipment. In addition to their use with ovarian cancer, the CNPs of the disclosure are contemplated to have utility with a broad range of disease states currently characterized with ultrasound imaging.

One aspect of the disclosure, therefore, encompasses embodiments of a method of generating a detectable photoacoustic signal in a biological subject, the method comprising: (i) administering to a biological subject a first pharmaceutically acceptable composition comprising a degradable cellulose nanoparticle probe that generates a detectable photoacoustic signal when exposed to an excitation optical energy; (ii) allowing the probe to concentrate in a target cell or tissue; (iii) allowing degradation of the administered cellulose nanoparticle probes when not concentrated in a target cell or tissue; (iv) illuminating the recipient subject with an incident energy having a wavelength that generates an emitted photoacoustic signal from the concentration of cellulose nanoparticle probes; (v) detecting the emitted photoacoustic signal; and (vi) determining at least one of the presence and the location of the emitted detectable photoacoustic signal in the biological subject.

In embodiments of this aspect of the disclosure, the method may further comprise generating from the detectable emitted photoacoustic signal an image of the concentration of the cellulose nanoparticle probes in the biological subject.

In embodiments of this aspect of the disclosure, the biological subject receiving the administered pharmaceutically acceptable composition can be an isolated cell, a cultured cell, an isolated tissue, or an animal or human subject.

In embodiments of this aspect of the disclosure, the degradation of the cellulose nanoparticle probes not concentrated in a target cell or tissue of the recipient subject increases the photoacoustic signal to background signal ratio.

In embodiments of this aspect of the disclosure, the cellulose nanoparticle probes not concentrated in target cell or tissue of the recipient subject can be degraded by an endogenous cellulase activity.

In some embodiments of this aspect of the disclosure, the method can further comprise delivering to the recipient subject a second pharmaceutically acceptable composition, wherein said composition can comprise a cellulase.

In embodiments of this aspect of the disclosure, the cellulase delivered to the recipient subject can be in an amount effective for the degradation of the cellulose nanoparticle probes not concentrated in target cell or tissue of the recipient subject.

In some embodiments of this aspect of the disclosure, the first and the second pharmaceutically acceptable compositions can be administered simultaneously to the recipient subject.

In some embodiments of this aspect of the disclosure, the first and the second pharmaceutically acceptable compositions can be administered consecutively to the recipient subject.

In embodiments of this aspect of the disclosure, the photoacoustic cellulose nanoparticles can have a fluorescent dye attached thereto.

In embodiments of this aspect of the disclosure, the photoacoustic cellulose nanoparticles can be adapted to selectively bind to a target cell or tissue by having a target-specific moiety attached thereto.

In embodiments of this aspect of the disclosure, the photoacoustic cellulose nanoparticles can be derived from cotton linter.

Another aspect of the disclosure encompasses embodiments of a degradable cellulose nanoparticle probe, wherein said nanoparticle can have an amorphous and anisotropic conformation having an average dimension of about 25 nanometers (nm) to about 250 nm.

In some embodiments of this aspect of the disclosure, the probe can absorb energy at about 700 nm.

In some embodiments of this aspect of the disclosure, the cellulose nanoparticle can have a diameter of about 130±45 nm.

In embodiments of this aspect of the disclosure, the nanoparticle can further comprise a dye compound or plurality of dye compounds attached to the cellulose nanoparticle.

In embodiments of this aspect of the disclosure, the dye can be selected from the group consisting of: a diarylrhodamine derivative, a polyaromatic-azo quencher, Blackberry Q, and a bisazulene derivative.

In embodiments of this aspect of the disclosure, the degradable cellulose nanoparticle probe can further comprise a targeting moiety bound to the cellulose nanoparticle.

In embodiments of this aspect of the disclosure, the targeting moiety can be bound to the cellulose nanoparticle via a linker molecule.

Still another aspect of the disclosure encompasses embodiments of a method of generating a degradable cellulose nanoparticle probe, the method comprising the steps of: contacting an aqueous suspension of cellulose fibers with about 6M sulfuric acid; cooling said acidified suspension to ambient temperature over a period of between about 2 h to about 4 h; fractionating the acidified suspension by centrifugation; resuspending a pelleted fraction from the centrifugation in water; and dialyzing the resuspended pellet with a dialysis membrane having a molecular weight cutoff value between about 3000 daltons to about 4000 daltons, thereby obtaining a suspension of cellulose nanoparticles having an amorphous and anisotropic conformation with an average dimension of about 25 nanometers (nm) to about 250 nm.

In embodiments of this aspect of the disclosure, the cellulose fibers can be from cotton linter.

Some embodiments of this aspect of the disclosure encompass cellulose nanoparticles generated by the method of disclosure.

Still another aspect of the disclosure encompasses embodiments of a kit that can comprise a container enclosing an amount of a degradable cellulose nanoparticle probe of the disclosure and instructions for obtaining a photoacoustic image of a biosample or animal or human subject using said probe.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

Reagents: The two sources of cellulose were cotton linters from Arnold Grummer Corp. and type 20 Sigmacell cellulose from Sigma. Cellulase from Aspergillus niger was purchased from Fisher and used without further purification. Modified Alamar Blue reagent (“Presto Blue”) was from Invitrogen. All water was purified to 18 MΩ and then filtered through a 0.2 μm filter.

### Example 2

Cellulose nanoparticle (CNP) Synthesis: The CNPs of the disclosure were made with a protocol adapted from Capadona et al., (2009) Biomacromolecules 10: 712-716, incorporated herein by reference in its entirety. Cellulose (500 mg) was added to 15 mL of water in an Erlenmeyer flask and stirred. 28 mL of 18 M sulfuric acid was then rapidly added and the exothermic reaction was allowed to cool back to room temperature over three hours. The material was then centrifuged for 10 min at 6,000 rpm, the supernatant decanted and the pellet re-suspended with distilled water. This content was then dialyzed with a 3500 molecular weight cutoff membrane (Pierce) for at least 24 h and then adjusted to pH 7 with diluted NaOH. The mass concentration was calculated by evaporating known volumes of CNPs at 90 ° C. overnight and weighing the dried samples.

### Example 3

Equipment: Size and zeta potential were obtained via dynamic light scattering (DLS) on a Zetasizer-90 instrument from Malvern Instruments (Worcestershire, UK). The measurements were made in 50% PBS/50% water. A Synergy 4 (Biotek) microplate reader was used for cell assays and absorbance measurements. All transmission electron microscopy (TEM) and energy-dispersive x-ray spectroscopy (EDS) was performed with a Tecnai G2 X-Twin (FEI Co.) instrument operating at 200 kV.

A Nexus 128 (Endra Life Sciences) was used for tomographic photoacoustic imaging. The Nexus uses an optical parametric oscillator (OPO) tunable laser and 128 detectors submerged in hemispherical bowl filled with water stabilized at 38° C. The animal or sample to be imaged was placed in a tray lying on top of the water in the center of the bowl. This tray contained a central indentation or dimple to immobilize a subcutaneous tumor or ex vivo sample for consistent spatial location.

Optimization scans used 60 views with 25 replicate pulses. Animal scans rotated the bowl through 120 views (3 degrees each) with 75 pulses per view with 8 min scan times. The incident radiation was selected by the operator prior during the scan setup. For spectral PA studies, a LAZR instrument (Visualsonics Corp.) was used for planar imaging. It was equipped with a 21 MHz-centered transducer as described by Needles et al., in Development of a Combined Photoacoustic Micro-Ultrasound System for Estimating Blood Oxygenation, IEEE: (2010); pp 390-393 and Jokerst et al., (2012) ACS Nano 5920-5930. This instrument also used an OPO laser operating at 20 Hz between 680 and 970 nm. Step sizes are 1 nm with 4-6 ns pulse width. The spot size is 1 mm×24 mm and the full field-of-view is 14-23 mm wide. Images were acquired at 5 frames per sec, and peak energy at the source is 45±5 mJ at 20 Hz.

### Example 4

Cell Culture and Animal Handling: In vivo imaging and in vitro studies used the OV2008 (also known as 2008) cell line. These cells were grown in DMEM supplemented with fetal bovine serum and antibiotics/antimycotics. Toxicity assays used a derivative of the Alamar Blue assay (Presto Blue). Here, 10,000 cells/well were plated and analyzed in replicate (n=8). Cells were exposed to increasing concentrations of CNPs for 18 h or 24 h after plating. Assay readout used 540 nm excitation and 600 nm emission.

Female nu/nu mice age 6-16 weeks were used and each data point included three mice unless otherwise noted. Before handling, animals were anesthetized with 2% isofluorane in oxygen at 1-3 L/min. To create subcutaneous xenograft tumors, 10⁷ cells in 50% growth factor-reduced matrigel/50% PBS were implanted into the hind limb of a nude mouse. Tumors were imaged when they reached 500 mm³, typically 1-2 weeks after implantation.

### Example 5

Biodegradation Experiments: These experiments followed established protocols (Moss & Bergmeyer Methods of Enzymatic Analysis. Academic Press: New York, N.Y., 1984; Vol. 2). This assay used hexokinase to catalyze a glucose phosphorylation from an ATP donor to give glucose-6-phosphate (G6P). G6P is then oxidized by nicotinamide adenine dinucleotide (NAD) to 6-phosphogluconate with glucose-6-phosphate dehydrogenase (G6PDH). An equivalent molecule of NAD is reduced to NADH with a subsequent change in absorbance at 340 nm (A340), which is directly proportional to glucose concentration. This assay was validated with a calibration curve as well as positive and negative controls. The calibration curve used glucose standards from 5-250 μg/mL. It was linear at R^{2>}0.999 and the relative standard deviation for each datum was less than 5%. The relationship between A340 and glucose concentration was used in subsequent experiments, as shown in FIG. 4A.

A solution containing 1.5 mM nicotinamide adenine dinucleotide (NAD), 1.0 mM ATP, 1.0 unit/ml of hexokinase, and 1.0 unit/ml of glucose-6-phosphate dehydrogenase was obtained from Sigma. CNPs and cellulose standards were brought to 1 mg/mL in 0.05 M acetic acid (pH=5.0). D-glucose standards (250-5 μg/mL) were prepared in the same acetic acid solution. Cellulase (5 U/mL) was prepared in cold distilled water. 4 mL of CNPs and controls were added to borosilicate test tubes followed by 1 mL of cellulase or water as a control. The solution was incubated at 37° C. with shaking. Aliquots were periodically removed and the cellulase activity quenched by placing the aliquots in an ice bath. The samples were centrifuged for 12 min at 12,000 rpm to removed unreacted materials. 40 μL aliquots of the supernatant and glucose standards were then placed in triplicate in a 96-well plate; 100 μL of the HK solution was added and allowed to react at room temperature for 15-17 min. Absorbance at 340 nm was measured and used to construct a standard curve and estimate available glucose.

### Example 6

Data Analysis: Photoacoustic data was reconstructed with a filtered backprojection algorithm proposed by Wang et al. (Wang et al., (2004) Phys. Med. Biol. 49: 3117). AMIDE.RTM software was used to create renderings of the images and all images were thresholded to the same value (Loening et al., (2003) Mol. Imaging 2: 131-137). To quantitate the images, MICROVIEW.RTM (General Electric Corp.) software was used. A region of interest (ROI) 15 mm×15 mm×15 mm was created around the sample and the mean intensity extracted. This intensity was assigned values of arbitrary units (a.u.) and used for the analysis and discussion below. The limit of detection (LOD; sensitivity) was defined as the concentration detectable 3 standard deviations above the signal of the blank. The time to half max (T½) is the time halfway between the pre-injection time point and maximum signal.

### Example 7

Statistical Treatment: To determine the average and standard deviations of data sets, the Excel functions “AVERAGE” and “STDEV” were used. Other metrics include the standard error of the mean that was computed by dividing the standard deviation by the square root of n samples. Relative standard deviation (RSD) was computed by dividing the standard deviation by the average. Significance testing used a two tailed, t-test through the “TTEST” function in Excel.

We claim the following:

1. A method of generating a detectable photoacoustic signal in a biological subject, the method comprising:
(i) administering to a biological subject a first pharmaceutically acceptable composition comprising a degradable cellulose nanoparticle probe that generates a detectable photoacoustic signal when exposed to an excitation optical energy;
(ii) allowing the probe to concentrate in a cell or tissue;
(iii) allowing degradation of the administered cellulose nanoparticle probes when not concentrated in a target cell or tissue;
(iv) illuminating the recipient subject with an incident energy having a wavelength that generates an emitted photoacoustic signal from the concentration of cellulose nanoparticle probes;
(v) detecting the emitted photoacoustic signal; and
(vi) determining at least one of the presence and the location of the emitted detectable photoacoustic signal in the biological subject.

2. The method of claim 1, further comprising generating from the detectable emitted photoacoustic signal a detectable image of the cellulose nanoparticle probes in the biological subject.

3. The method of claim 1, wherein the biological subject receiving the administered pharmaceutically acceptable composition is an isolated cell, a cultured cell, an isolated tissue, or, a tissue or region of an animal or human subject.

4. The method of claim 1, wherein the degradation of the cellulose nanoparticle probes not concentrated in a target cell or tissue of the recipient subject increases the photoacoustic signal to background signal ratio.

5. The method of claim 1, the method further comprising allowing the cellulose nanoparticle probes degrade after step (v).

6. The method of claim 1, wherein the cellulose nanoparticle probes are degraded by an endogenous cellulase activity.

7. The method of claim 1, further comprising delivering to the recipient subject a second pharmaceutically acceptable composition, wherein said composition comprises a cellulase.

8. The method of claim 7, wherein the cellulase delivered to the recipient subject is in an amount effective for the degradation of the cellulose nanoparticle probes not concentrated in target cell or tissue of the recipient subject.

9. The method of claim 7, wherein the first and the second pharmaceutically acceptable compositions are administered simultaneously to the recipient subject.

10. The method of claim 7, wherein the first and the second pharmaceutically acceptable compositions are administered consecutively to the recipient subject.

11. The method of claim 1, wherein the photoacoustic cellulose nanoparticles have a fluorescent dye attached thereto.

12. The method of claim 1, wherein the photoacoustic cellulose nanoparticles are adapted to selectively bind to a target cell or tissue by having a target-specific moiety attached thereto.

13. The method of claim 1, wherein the photoacoustic cellulose nanoparticles are derived from cotton linter.

14. A degradable cellulose nanoparticle probe, wherein said nanoparticle has an amorphous and anisotropic conformation having an average dimension of about 25 nanometers (nm) to about 250 nm.

15. The degradable cellulose nanoparticle probe of claim 14, wherein the probe absorbs energy at about 700 nm.

16. The degradable cellulose nanoparticle probe of claim 15, wherein the cellulose nanoparticle has a diameter of about 130±45 nm.

17. The degradable cellulose nanoparticle probe of claim 14, wherein said nanoparticle further comprises a dye compound or plurality of dye compounds attached to the cellulose nanoparticle.

18. The degradable cellulose nanoparticle probe of claim 17, wherein the dye is selected from the group consisting of: a diarylrhodamine derivative, a polyaromatic-azo quencher, Blackberry Q, and a bisazulene derivative.

19. The degradable cellulose nanoparticle probe of claim 14, further comprising a targeting moiety bound to the cellulose nanoparticle.

20. The degradable cellulose nanoparticle probe of claim 19, wherein the targeting moiety is bound to the cellulose nanoparticle via a linker molecule.

21. A method of generating a degradable cellulose nanoparticle probe, the method comprising the steps of: contacting an aqueous suspension of cellulose fibers with about 6M sulfuric acid; cooling said acidified suspension to ambient temperature over a period of between about 2 h to about 4 h; fractionating the acidified suspension by centrifugation; resuspending a pelleted fraction from the centrifugation in water; and dialyzing the resuspended pellet with a dialysis membrane having a molecular weight cutoff value between about 3000 daltons to about 4000 daltons, thereby obtaining a suspension of cellulose nanoparticles having an amorphous and anisotropic conformation with an average dimension of about 25 nanometers (nm) to about 250 nm.

22. The method of claim 21, wherein the cellulose fibers are from cotton linter.

23. A cellulose nanoparticle generated by the method of claim 21.

24. A kit comprising a container enclosing an amount of a degradable cellulose nanoparticle probe of the disclosure and instructions for obtaining a photoacoustic image of a biosample or animal or human subject using said probe.
